(19) United States
(12) Patent Application Publication
(10) Pub. No.: US 2026/0026730 A1
(43) Pub. Date: Jan. 29, 2026

(54) BIOSIGNAL MEASUREMENT SYSTEM

(71) Applicant: NTT, Inc., Tokyo (JP)

(72) Inventors: Kenichi Matsunaga, Tokyo (JP); Kento Watanabe, Tokyo (JP)

(21) Appl. No.: 19/107,913

(22) PCT Filed: Sep. 1, 2022
(86) PCT No.: PCT/JP2022/032935

§ 371 Date: Feb. 28, 2025

(51) Int. Cl. A61B 5/308; A61B 5/00; A61B 5/28; A61B 5/305; A61B 5/327; A61B 5/346

(52) U.S. Cl. CPC A61B 5/308; A61B 5/0024; A61B 5/28; A61B 5/305; A61B 5/327; A61B 5/346; A61B 2560/0214

(57) ABSTRACT

An embodiment is a biosignal measurement system including two electrode devices, a biosignal generation device, and a right-leg drive device. Each electrode device has a first electrode, a non-inverting amplification circuit, a quantization circuit, a first wireless transmitter, an FM transmitter, an FM receiver, an adjustment circuit, and a power supply. The biosignal generation device has a first wireless receiver, an arithmetic circuit, a midpoint potential calculation circuit, and a second wireless transmitter. The right-leg drive device has a second wireless receiver, an amplifier circuit, and a second electrode. The electrode devices measure and process biopotentials, transmitting information wirelessly and via FM signals. The biosignal generation device receives this information, generates waveforms, calculates midpoint potentials, and transmits them to the right-leg drive device, which applies the amplified potential to the body.

### CROSS-REFERENCE TO RELATED APPLICATIONS

This application is a national phase entry of PCT Application No. PCT/JP2022/032935, filed on Sep. 1, 2022, which application is hereby incorporated herein by reference.

### TECHNICAL FIELD

The present invention relates to a biosignal measurement system.

### BACKGROUND

In electrocardiogram measurement, which is one of biopotential measurements, a potential difference between electrodes disposed on both left and right sides of the human body is measured. For example, as illustrated in FIG. 5, there has been proposed a measurement system in which a device 301 is attached to a central portion of a body, and an electrode 304 is provided to contact with left and right waist portions by causing a wire 303 to extend along a compression wear 302 (Non Patent Literature 1).

Furthermore, in the biopotential measurement, removal of common mode noise is an important factor for stable operation, and as illustrated in FIG. 6, a right-leg drive device (Right Leg Drive; RLD) circuit that feeds back a midpoint potential 401 of a differential voltage and controls the midpoint potential 401 to be an arbitrary voltage is often implemented (Non Patent Literature 2).

### CITATION LIST

### Non Patent Literature

Non Patent Literature 1: Nahoko Kasai and others, “Development of Functional Textile “hitoe”: Wearable Electrodes for Monitoring Human Vital Signals” Communication Society Magazine, 2017, Vol. 11, No. 1, pp.17-23, The Institute of Electronics, Information and Communication Engineers.
Non Patent Literature 2: Analog Devices, Inc., Technical article MS-2385, “Jibun no genkai wo yosoku shi kakunin shiyou! (in Japanese) (Let's predict your limitations and confirm this!), [retrieved on Aug. 19, 2022], (https://www.analog.com/media/jp/technical-documentation/tech-articles/ms-2385_jp.pdf).

### SUMMARY

### Technical Problem

However, attachment on the body causes a feeling of discomfort due to a feeling of pressure and a feeling of avoidance due to taking labor for attachment. Other than the body, for example, it is conceivable to attachment to four limbs. However, in this case, since the wire connecting the left and right electrodes forms a loop like handcuffs, it strongly restricts the movement of the body. These problems can be solved as long as the wire between the left and right electrodes can be cut and the devices can be separated into two units, but the reference of potential measurement cannot be determined through the separation, and it becomes difficult to perform the biopotential measurement. Furthermore, since the right-leg drive device is often attached to the right leg as the name of this, the number of wires to be laid on the living body is further increased, and discomfort is induced.

Embodiments of the present invention has been made to solve the above-described problems, and an object thereof is to enable a biopotential to be easily measured even when the wire between two electrodes and the right-leg drive device is cut and the devices are separated into three units.

### Solution to Problem

According to embodiments of the present invention, there is provided a biosignal measurement system including two electrode devices, a right-leg drive device, and a biosignal generation device, in which each of the two electrode devices includes: a first electrode that measures a biopotential in a target human body; a non-inverting amplification circuit that inputs the measured biopotential to a non-inverting amplifier terminal, amplifies the biopotential, and outputs the amplified biopotential from an output terminal; a quantization circuit that converts an amplified signal output from the output terminal of the non-inverting amplifier circuit into digital data and generates biopotential information; a first wireless transmitter that transmits the biopotential information to the biosignal generation device; an FM transmitter that converts a voltage signal output from the output terminal of the non-inverting amplifier circuit into an FM signal and transmits the FM signal to the other electrode device; an FM receiver that receives the FM signal transmitted from the other electrode device to an own electrode device, converts the FM signal into a voltage signal, and outputs the voltage signal; an adjustment circuit that outputs the voltage signal output from the FM receiver to an inverting input terminal of the non-inverting amplifier circuit as an adjustment signal adjusted under a set condition; and a power supply that supplies power to the non-inverting amplifier circuit, the quantization circuit, the first wireless transmitter, the FM transmitter, the FM receiver, and the adjustment circuit, a frequency of the FM signal transmitted from one of the two electrode devices to the other electrode device and a frequency of the FM signal transmitted from the other electrode device to the one electrode device have different frequencies, the biosignal generation device includes a first wireless receiver that receives the biopotential information transmitted from each of the two electrode devices, an arithmetic circuit that generates a biosignal waveform by using the biopotential information received by the first wireless receiver, a midpoint potential calculation circuit that obtains a midpoint potential from the biopotential information received by the first wireless receiver and transmitted from each of the two electrode devices, and a second wireless transmitter that wirelessly transmits the midpoint potential to the right-leg drive device, and the right-leg drive device includes a second wireless receiver that receives the midpoint potential transmitted from the second wireless transmitter, an amplifier circuit that amplifies the midpoint potential received by the second wireless receiver, and a second electrode that applies the midpoint potential amplified by the amplifier circuit to the human body.

### Advantageous Effects

As described above, according to embodiments of the present invention, two electrode devices and the biosignal generation device are connected by wireless communication, and the two electrode devices are connected by FM communication, and the right-leg drive device and the biosignal generation device are connected by the wireless communication. Therefore, even when the wire between the two electrodes and the right-leg drive device is cut and the devices are separated into three units, the biopotential can be easily measured.

### BRIEF DESCRIPTION OF THE DRAWINGS

FIG. 1A is a configuration diagram illustrating a configuration of a biosignal measurement system according to a first embodiment of the present invention.

FIG. 1B is a configuration diagram illustrating a partial configuration of a biosignal measurement system according to the first embodiment of the present invention.

FIG. 2 is an explanatory diagram illustrating a concept of a biosignal measurement system according to the first embodiment of the present invention.

FIG. 3 is a configuration diagram illustrating a configuration of another biosignal measurement system according to the first embodiment of the present invention.

FIG. 4 is a configuration diagram illustrating a configuration of a biosignal measurement system according to a second embodiment of the present invention.

FIG. 5 is a configuration diagram illustrating a configuration of a biosignal measurement system of the related art.

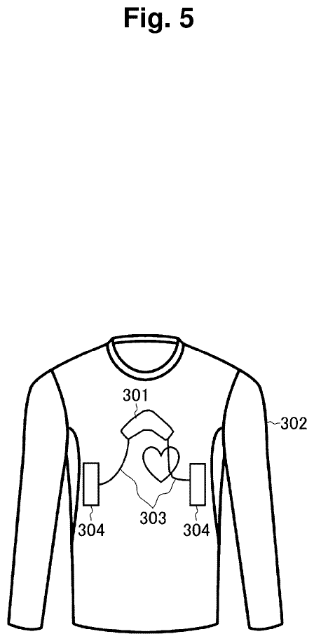

FIG. 6 is a configuration diagram illustrating a configuration of a biosignal measurement system of the related art using a right-leg drive device.

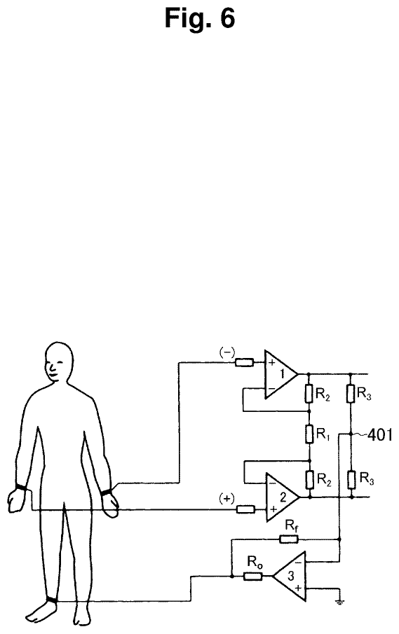

### DETAILED DESCRIPTION OF ILLUSTRATIVE EMBODIMENTS

A biosignal measurement system according to an embodiment of the present invention will be described below.

### First embodiment

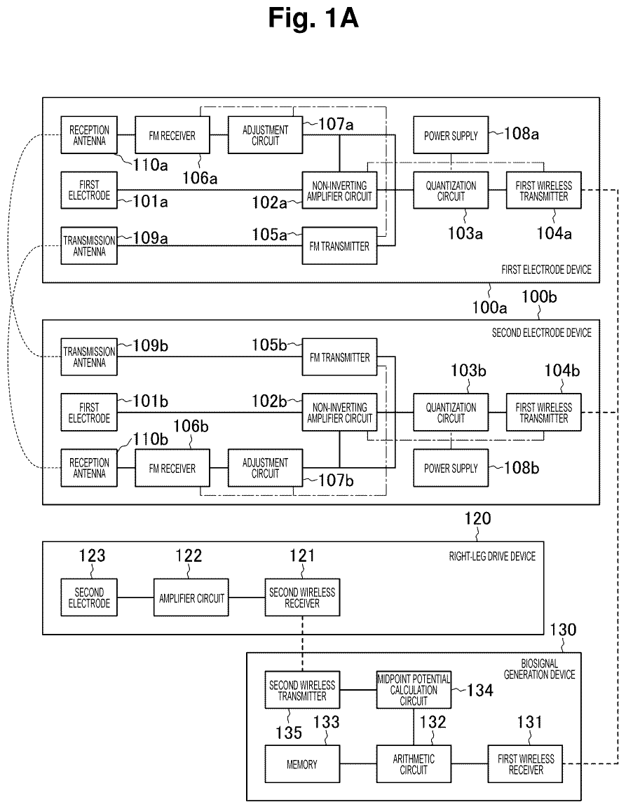

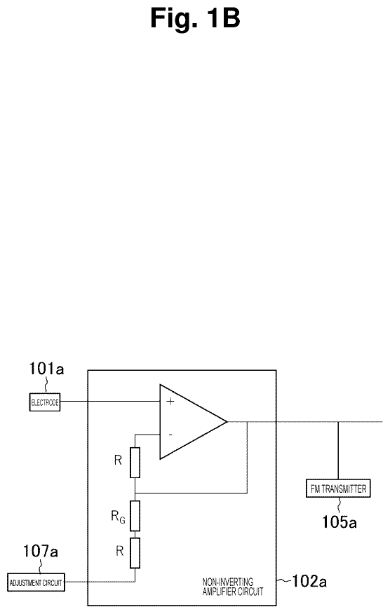

First, a biosignal measurement system according to a first embodiment of the present invention will be described with reference to FIGS. 1A and 1B. This system includes two of a first electrode device 100a and a second electrode device 100b, a right-leg drive device 120 and a biosignal generation device 130.

First, the first electrode device 100a includes an electrode 101a that measures a biopotential in a target human body, a non-inverting amplifier circuit 102a that inputs the measured biopotential to a non-inverting amplifier terminal, amplifies the biopotential, and outputs the amplified biopotential from an output terminal, a quantization circuit 103a that converts the amplified signal output from the output terminal of the non-inverting amplifier circuit 102a into digital data to generate biopotential information, and a first wireless transmitter 104a that transmits the biopotential information to the biosignal generation device 130.

First, the first electrode device 100a includes an FM transmitter 105a, an FM receiver 106a, and an adjustment circuit 107a. The FM transmitter 105a converts a voltage signal output from the output terminal of the non-inverting amplifier circuit 102a into an FM signal, and transmits the converted FM signal to the second electrode device 100b through a transmission antenna 109a. The FM receiver 106a converts the FM signal transmitted from the second electrode device 100b to the first electrode device 100a and received through a reception antenna 110a into a voltage signal and outputs the voltage signal. In the first electrode device 100a, the adjustment circuit 107a outputs the voltage signal output from the FM receiver 106a as an adjustment signal adjusted in a set condition to an inverting input terminal of the non-inverting amplifier circuit 102a.

The output of the non-inverting amplifier circuit 102a is also input to the inverting input terminal. For example, as illustrated in FIG. 1B, when a signal input from the adjustment circuit 107a to the inverting input terminal of the non-inverting amplifier circuit 102a is Vdev2 and an output of the non-inverting amplifier circuit 102a is Vout1 at a negative terminal input Vin− of an operational amplifier of the non-inverting amplifier circuit 102a, the signals are mixed at a ratio of “Vin−=(R+RG)/(2R+RG)Vout+(R)/(2R+RG)Vdev2” and input to the inverting input terminal of the non-inverting amplifier circuit 102a. The same applies to a non-inverting amplifier circuit 102b to be described later.

Furthermore, the first electrode device 100a includes a power supply 108a that supplies power to the non-inverting amplifier circuit 102a, the quantization circuit 103a, the first wireless transmitter 104a, the FM transmitter 105a, the FM receiver 106a, and the adjustment circuit 107a.

First, the second electrode device 100b includes an electrode 101b that measures a biopotential in a target human body, the non-inverting amplifier circuit 102b that inputs the measured biopotential to a non-inverting amplifier terminal, amplifies the biopotential, and outputs the amplified biopotential from an output terminal, a quantization circuit 103b that converts the amplified signal output from the output terminal of the non-inverting amplifier circuit 102b into digital data to generate biopotential information, and a first wireless transmitter 104b that transmits the biopotential information to the biosignal generation device 130.

Furthermore, the second electrode device 100b includes an FM transmitter 105b, an FM receiver 106b, and an adjustment circuit 107b. The FM transmitter 105b converts a voltage signal output from the output terminal of the non-inverting amplifier circuit 102b into an FM signal, and transmits the converted FM signal to the first electrode device 100a through a transmission antenna 109b. The FM receiver 106b converts the FM signal transmitted from the first electrode device 100a to the second electrode device 100b and received through a reception antenna 110b into a voltage signal and outputs the voltage signal. The adjustment circuit 107b outputs the voltage signal output from the FM receiver 106b as an adjustment signal adjusted in a set condition to an inverting input terminal of the non-inverting amplifier circuit 102b.

Furthermore, the second electrode device 100b includes a power supply 108b that supplies power to the non-inverting amplifier circuit 102b, the quantization circuit 103b, the first wireless transmitter 104b, the FM transmitter 105b, the FM receiver 106b, and the adjustment circuit 107b.

Here, the frequency of the FM signal transmitted from the first electrode device 100a to the second electrode device 100b is different from the frequency of the FM signal transmitted from the second electrode device 100b to the first electrode device 100a.

The biosignal generation device 130 includes a first wireless receiver 131 that receives the biopotential information transmitted from each of the first electrode device 100a and the second electrode device 100b, and an arithmetic circuit 132 that generates a biosignal waveform by using the biopotential information received by the first wireless receiver 131. For example, the arithmetic circuit 132 can generate an electrocardiogramal waveform by using two pieces of biopotential information transmitted from each of the first electrode device 100a and the second electrode device 100b, which are attached to any two positions of four limbs of the human body. Furthermore, the biosignal generation device 130 includes a memory 133 that stores the biosignal waveform generated by the arithmetic circuit 132.

Moreover, the biosignal generation device 130 includes a midpoint potential calculation circuit 134 that obtains a midpoint potential from the biopotential information received by the first wireless receiver 131 and transmitted from each of two of the first electrode devices 100a and the second electrode device 100b, and a second wireless transmitter 135 that wirelessly transmits the midpoint potential to the right-leg drive device 120.

The right-leg drive device 120 includes a second wireless receiver 121 that receives the midpoint potential transmitted from the second wireless transmitter 135, an amplifier circuit 122 that amplifies the midpoint potential received by the second wireless receiver 121, and a second electrode 123 that applies the midpoint potential amplified by the amplifier circuit 122 to the human body.

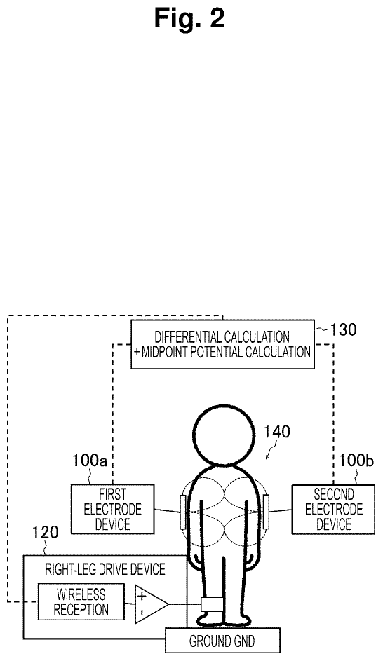

FIG. 2 illustrates a concept of the biosignal measurement system according to the first embodiment. The biosignal generation device 130 obtains a midpoint potential from the biopotential information measured by the first electrode device 100a and the second electrode device 100b, which are attached to a human body 140. The obtained midpoint potential is wirelessly transmitted to the right-leg drive device 120 attached to the human body 140 to be fed back. As a result, it is possible to perform stable biopotential measurement while preventing discomfort from being induced due to the increase in the number of wires. According to this configuration, it is possible to suppress common mode noise, and thus, stable biopotential measurement can be performed.

In this example, the non-inverting amplifier circuit 102a of the first electrode device 100a and the non-inverting amplifier circuit 102b of the second electrode device 100b are coupled to each other. Since such a configuration is similar to the configuration of an oscillation circuit, oscillation occurs when the phase rotation is 180 degrees and the amplification degree is one or more in the phase rotation and amplification degree caused by the delay of the signals to be coupled with each other.

As an example, in a case where a non-inverting amplifier circuit typically having a bandwidth of DC to 1 kHz in the biosignal is built, when a delay of 0.1 ms occurs, the phase rotation of 36 degrees occurs in a 1 kHz signal. That is, since the phase rotation of 180 degrees occurs with a delay of 0.5 ms, the possibility of oscillation cannot be denied. Therefore, in the mutual coupling between the non-inverting amplifier circuit 102a and the non-inverting amplifier circuit 102b described above, it is necessary to minimize the delay at a portion to which the voltage signal is coupled.

Next, the electrode 101a and the electrode 101b will be described. As these electrodes, various electrodes can be used, and any electrodes such as an Ag/AgCl electrode used also in medical application, a conductive cloth electrode, and a metal electrode can be used. In particular, it is also possible to further improve the usability by adopting a non-contact electrode configuration in which a sensor device is attached on the clothes by using a cloth or metal electrode that does not need to be adhered to the human body. In particular, for the non-contact electrode configuration, capacitive coupling is suitable since it is easy to pass through in high frequency communication.

Next, the adjustment circuit 107a and the adjustment circuit 107b will be described. These are portions that perform adjustment by a constant multiplication on the basis of the received FM signal, and thus can be configured by the operational amplifier. Although the operational amplifiers can be connected in multiple stages, the delay is accumulated every time the operational amplifiers are connected in multiple stages, and thus the operational amplifiers are likely to be unstable. Therefore, it is preferable that each of the adjustment circuit 107a and the adjustment circuit 107b comprises by a minimum one-stage operational amplifier.

Next, the non-inverting amplifier circuit 102a and the non-inverting amplifier circuit 102b will be described. Since the biopotential is a very weak signal, signal amplification by the non-inverting amplifier circuit 102a and the non-inverting amplifier circuit 102b each configured by a filter circuit and an amplifier circuit configured by an operational amplifier is required. By adopting the non-inverting amplifier circuit, it is possible to realize a configuration equivalent to that of an instrumentation amplifier having a high common mode suppression capability as a system.

Furthermore, in the amplification stages of the non-inverting amplifier circuit 102a and the non-inverting amplifier circuit 102b, a high input impedance is required in order to reduce the loss of the biopotential, but even when the non-inverting amplifier circuit 102a and the non-inverting amplifier circuit 102b have a high input impedance configuration, the noise hardly increases. On the other hand, with an inverting amplifier circuit, the resistance for determining the input impedance also affects the gain setting, and further directly contributes as thermal noise, so that the SN ratio is lowered. Therefore, the non-inverting amplifier circuit is effective.

Since the potential difference between the two electrodes is detected in the biopotential measurement, the same reference potential is required in the non-inverting amplifier circuit 102a and the non-inverting amplifier circuit 102b. Therefore, by using the potentials generated by the adjustment circuits 107a and the adjustment circuit 107b, balanced signal amplification between the first electrode devices 100a and the second electrode device 100b is possible, and good biosignal information is finally obtained.

Furthermore, the FM communication frequency used when the non-inverting amplifier circuit 102a and the non-inverting amplifier circuit 102b are coupled to each other needs to have different frequencies. This is because when the same frequency is used, mutual interference occurs and desired coupling cannot be obtained. This corresponds to dividing a band in communication, and by increasing the frequency to be used, embodiments of the present invention can be used not only in a configuration in which electrode devices are paired, but also among more electrode devices.

Next, the first wireless transmitter 104a and the first wireless transmitter 104b will be described. For example, the first wireless transmitter 104a may be only required to be configured by one communication module, and may be only required to be connected so as to be capable of receiving a measurement potential output from the quantization circuit 103a and transmitting the measurement potential to the biosignal generation device 130.

As a standard of a wireless communication network among the first wireless transmitter 104a, the first wireless transmitter 104b, and the first wireless receiver 131, any standards such as carrier communication, Wi-Fi (registered trademark), and Bluetooth (registered trademark) can be applied. It is necessary to select a transmitter and a receiver according to a communication standard. In a short-range communication standard such as Bluetooth, a smartphone or the like which is a terminal close to a user, which is a human body to be measured, can be used as the biosignal generation device 130. Furthermore, when Wi-Fi or the like is used, a server or the like can be used as the biosignal generation device 130.

Furthermore, functions required by the biosignal generation device 130 are to receive signals from a plurality of electrode devices and to obtain a target biopotential by calculation. These functions can be implemented (incorporated) in any electrode device without using the biosignal generation device 130. In this case, for example, the second electrode device 100b is provided to which the biosignal generation device including an arithmetic circuit, a memory, a midpoint potential calculation circuit, and a second wireless transmitter is added.

In this case, the second electrode device 100b includes a first wireless receiver instead of the first wireless transmitter, receives the biopotential information transmitted from the first electrode device 100a, and obtains the biopotential by calculating the biopotential information together with the biopotential information from the second electrode device 100b. Furthermore, the midpoint potential calculation circuit obtains a midpoint potential, and the second wireless transmitter wirelessly transmits the obtained midpoint potential to the right-leg drive device 120. Furthermore, by storing the obtained biopotential in the memory 133 of the second electrode device 100b, the same function and effect as described above can be achieved. In addition, in this configuration, since it is not necessary to separately provide the biosignal generation device 130, it is not necessary to carry the smartphone or the like, and it is possible to implement measurement without limitation for the user.

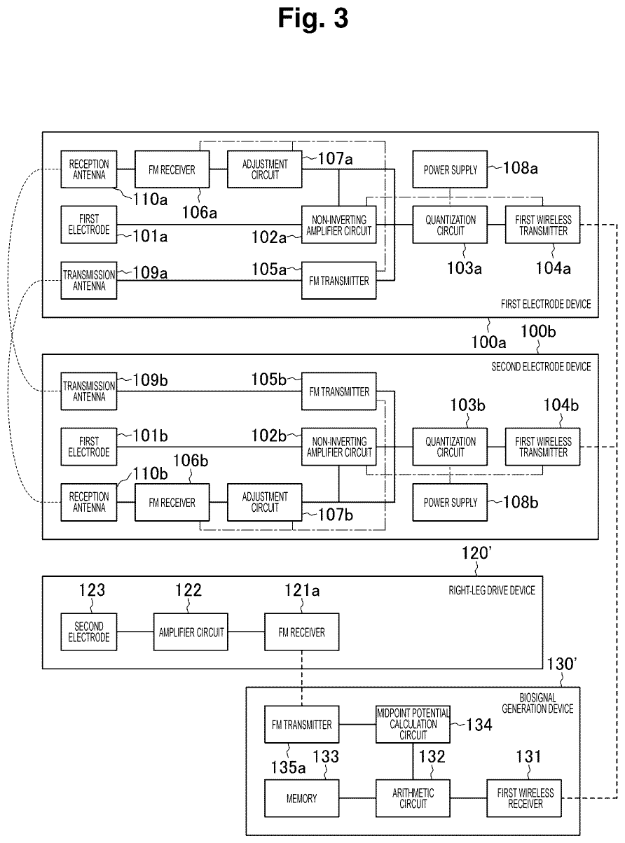

Furthermore, the second wireless transmitter can transmit the midpoint potential to the second wireless receiver by FM communication. This configuration will be described with reference to FIG. 3. As illustrated in FIG. 3, a biosignal generation device 130′ transmits the midpoint potential obtained by the midpoint potential calculation circuit 134 to a right-leg drive device 120′ through a FM transmitter 135a, and the right-leg drive device 120′ receives the transmitted midpoint potential through a FM receiver 121a. The other configurations are similar to those described above.

Furthermore, at least one of communication between two electrode devices or communication between the biosignal generation device and the right-leg drive device can be performed using a human body as a communication channel. In this case, in the electrode device, for example, a transmission electrode can be used instead of the transmission antenna, and a reception electrode can be used instead of the reception antenna.

By performing FM communication via the human body, delay and power are reduced, and the human body functions as a waveguide. Therefore, there is an advantage that radio waves can be confined, interference from the outside is strong, and a risk of causing interference to the outside can be reduced. Also in a case where the human body is transmitted, the frequency of the FM signal transmitted from the first electrode device 100a to the second electrode device 100b is different from the frequency of the FM signal transmitted from the second electrode device 100b to the first electrode device 100a. By setting the frequency band to be used to about several MHz to 100 MHz on the basis of the electrical properties of the human body, less loss can be achieved.

Furthermore, in a case where the human body is used as the communication channel, in the electrode device, three of the first electrode, the transmission electrode, and the reception electrode are used, but since the frequencies are different from each other, it is possible to provide one electrode by providing a band-pass filter, and there is an effect of improving the comfort of the user since the portion to be brought into contact with the human body is reduced.

Furthermore, in a case where the human body is used as a communication channel, the impedance of the human body changes, and the degree of contact between the electrode and the human body also changes. Therefore, in a system such as AM or PM modulation, it is not possible to prevent noise generation caused by amplitude fluctuation. On the other hand, in the FM communication, the noise to the amplitude does not affect the output. That is, the FM communication is suitable for a case where the human body is used as the communication channel.

### Second Embodiment

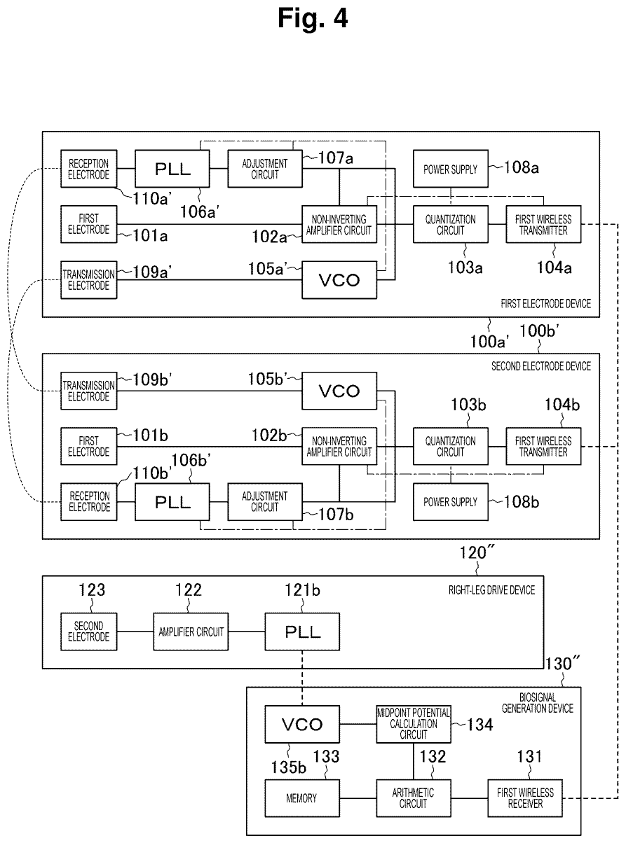

Next, a biosignal measurement system according to a second embodiment of the present invention will be described with reference to FIG. 4. This system includes two of a first electrode device 100a′ and a second electrode device 100b′, a right-leg drive device 120″ and a biosignal generation device 130″.

In the second embodiment, as the FM transmitter, a voltage control oscillator (VCO 105a′, VCO 105b′, VCO 135b) is used, and as the FM receiver, a phase locked loop (PLL 106a′, PLL 106b′, PLL 121b) is used. The other configurations are similar to the configuration in the case where the human body described above is used as the channel, and in the second embodiment, the transmission electrode 109a′, the transmission electrode 109b′, the reception electrode 110a′, and the reception electrode 110b′ are used.

In the configuration in which the FM communication is implemented using the human body described in the first embodiment as the communication channel, there is a possibility that a parameter related to the delay greatly varies. Therefore, it is necessary to use a device having a small delay particularly at the time of transmission and reception in the FM communication, and as an example of this, first, the FM transmitter comprises the voltage control oscillator, and the output frequency is directly modulated by the voltage. Furthermore, the FM receiver comprises the phase locked loop, and set as a direct detection system.

In a case where the human body is used as a communication channel (communication path), a high SN can be expected due to a radio wave confinement effect. Therefore, in a case where the human body is used as the channel, it is effective to employ a configuration with less delay than demodulation with high accuracy. The delay can be effectively reduced by configuring the FM transmitter as the voltage control oscillator and the FM receiver as the phase locked loop.

By the way, since the FM transmitter comprises as the voltage control oscillator, and the FM receiver comprises as the phase locked loop, the delay can be reduced. However, in a case where direct detection and direct modulation are adopted, it is essential for communication with less error that the voltage-to-frequency conversion characteristics match between the voltage control oscillator constituting the FM transmitter and the voltage control oscillator included in the phase locked loop constituting the FM receiver. However, the voltage control oscillator uses LC resonance caused by a variable capacitance diode generally called a varactor or oscillation caused by a ring oscillator. Due to manufacturing variations of these elements, the voltage-to-frequency conversion characteristics may not match between the voltage control oscillator constituting the FM transmitter and the voltage control oscillator included in the phase locked loop constituting the FM receiver.

As described above, as an example in which the voltage-frequency conversion characteristics do not match, in a case where the center frequencies do not match, it is possible to perform adjustment by adding an offset to the operational amplifier included in the adjustment circuit so that the voltage-frequency characteristics between the voltage control oscillator constituting the FM transmitter of the other electrode device and the voltage control oscillator of the phase locked loop of the own electrode device match. For example, the offset can be determined on the basis of the input voltage to the operational amplifier. In this manner, in a case where the FM communication is adopted, it is possible to perform adjustment without increasing the delay.

Furthermore, as an example in which the voltage-frequency conversion characteristics do not match, in a case where the slopes of the voltage-frequency characteristics do not match, the oscillation frequency characteristics are monitored, and it is possible to perform adjustment without increasing the delay by adjusting an amplification condition of the operational amplifier included in the adjustment circuit so that the voltage-frequency characteristics between the voltage control oscillator constituting the FM transmitter of the other electrode device and the voltage control oscillator of the phase locked loop of the own electrode device match. For example, the amplification condition of the operational amplifier can be adjusted by making the resistance value of the operational amplifier variable.

As described above, by appropriately adjusting the condition of the operational amplifier in the adjustment circuit, it is possible to cover the weak point in the FM communication, which is caused by the voltage control oscillator and the phase locked loop. In particular, in a case where the human body is used as the communication channel, the above-described configuration is suitable.

As described above, according to embodiments of the present invention, two electrode devices and the biosignal generation device are connected by wireless communication, and the two electrode devices are connected by FM communication, and the right-leg drive device and the biosignal generation device are connected by the wireless communication. Therefore, even when the wire between the two electrodes and the right-leg drive device is cut and the devices are separated into three units, the biopotential can be easily measured.

Note that the present invention is not limited to the above-described embodiments, and it is apparent to those skilled in the art that many modifications and combinations can be made within the technical idea of the present invention.

### REFERENCE SIGNS LIST

100a First electrode device
100b Second electrode device
101a, 101b Electrode
102a, 102b Non-inverting amplifier circuit
103a, 103b Quantization circuit
104a, 104b First wireless transmitter
105a, 105b FM transmitter
106a, 106b FM receiver
107a, 107b Adjustment circuit
108a, 108b Power supply
109a, 109b Transmission antenna
110a, 110b Reception antenna
120 Right-leg drive device
121 Second wireless receiver
123 Second electrode
130 Biosignal generation device
131 First wireless receiver
132 Arithmetic circuit
133 Memory
134 Midpoint potential calculation circuit
135 Second wireless transmitter

1-7. (canceled)

8. A biosignal measurement system comprising two electrode devices, a right-leg drive device, and a biosignal generation device,
wherein each of the two electrode devices includes:
a first electrode for measuring a biopotential in a target human body;
a first amplification circuit including a non-inverting input terminal, an inverting input terminal and an output terminal, the first amplification circuit configured to amplify the biopotential received via the first electrode by the non-inverting input terminal, and output an amplified signal from the output terminal;
a quantization circuit configured to convert the amplified signal output from the output terminal of the first amplification circuit into digital data to generate biopotential information;
a first wireless transmitter for transmitting the biopotential information to the biosignal generation device;
an FM transmitter configured to convert the amplified signal output from the output terminal of the first amplification circuit into an FM signal and transmit the FM signal to the other electrode device;
an FM receiver configured to receive another FM signal transmitted from the other electrode device, convert the other FM signal into a voltage signal, and output the voltage signal;
an adjustment circuit configured to adjust the voltage signal output from the FM receiver under a set condition and output the voltage signal adjusted under the set condition to the inverting input terminal of the first amplification circuit as an adjustment signal; and
a power supply for supplying power to the first amplification circuit, the quantization circuit, the first wireless transmitter, the FM transmitter, the FM receiver, and the adjustment circuit,
the biosignal generation device includes
a first wireless receiver configured to receive the biopotential information transmitted from each of the two electrode devices,
an arithmetic circuit configured to generate a biosignal waveform by using the biopotential information received by the first wireless receiver,
a midpoint potential calculation circuit configured to obtain a midpoint potential from the biopotential information transmitted from each of the two electrode devices and received by the first wireless receiver, and
a second wireless transmitter configured to transmit wirelessly the midpoint potential to the right-leg drive device, and
the right-leg drive device includes
a second wireless receiver configured to receive the midpoint potential transmitted from the second wireless transmitter,
a second amplification circuit configured to amplify the midpoint potential received by the second wireless receiver, and
a second electrode configured to apply the midpoint potential amplified by the second amplification circuit to the human body.

9. The biosignal measurement system according to claim 8,
wherein the second wireless transmitter transmits the midpoint potential to the second wireless receiver by FM communication.

10. The biosignal measurement system according to claim 8,
wherein one of the two electrode devices incorporates the biosignal generation device.

11. The biosignal measurement system according to claim 8,
wherein at least one of communication between the two electrode devices and communication between the biosignal generation device and the right-leg drive device is provided using the human body as a communication channel.

12. The biosignal measurement system according to claim 8,
wherein at least one of the FM transmitter and the second wireless transmitter comprises a voltage control oscillator, and
at least one of the FM receiver and the second wireless transmitter comprises a phase locked loop.

13. The biosignal measurement system according to claim 12,
wherein the adjustment circuit includes an operational amplifier, and is configured perform at least one of adjustment of an amplification condition of the operational amplifier and adding offset to the operational amplifier so that a voltage-frequency characteristic of the voltage control oscillator of the other electrode device and a voltage-frequency characteristic of a voltage control oscillator of the phase locked loop of the electrode device match with each other.

14. The biosignal measurement system according to claim 8,
wherein the arithmetic circuit generates an electrocardiogramaignal waveform by using two pieces of the biopotential information transmitted from each of the two electrode devices attached to any two positions of four limbs of the human body.

15. The biosignal measurement system according to claim 8,
wherein a frequency of the FM signal transmitted from one of the two electrode devices to the other electrode device and a frequency of the FM signal transmitted from the other electrode device to the one electrode device have different frequencies.

16. A biosignal measurement system comprising:
a first electrode device and a second electrode device, each electrode device comprising:
a first electrode configured to measure a biopotential in a target human body;
a non-inverting amplification circuit configured to amplify the measured biopotential;
a quantization circuit configured to convert an amplified signal from the non-inverting amplification circuit into digital biopotential information;
a first wireless transmitter configured to transmit the biopotential information;
an FM transmitter configured to convert the amplified signal into an FM signal and transmit the FM signal to the other electrode device;
an FM receiver configured to receive an FM signal from the other electrode device and convert it to a voltage signal;
an adjustment circuit configured to adjust the voltage signal and output it to the non-inverting amplification circuit; and
a power supply;
a biosignal generation device comprising:
a first wireless receiver configured to receive biopotential information from the electrode devices;
an arithmetic circuit configured to generate a biosignal waveform using the received biopotential information;
a midpoint potential calculation circuit configured to obtain a midpoint potential from the received biopotential information; and
a second wireless transmitter configured to wirelessly transmit the midpoint potential; and
a right-leg drive device comprising:
a second wireless receiver configured to receive the midpoint potential;
an amplifier circuit configured to amplify the received midpoint potential; and
a second electrode configured to apply the amplified midpoint potential to the human body.

17. The biosignal measurement system of claim 16, wherein the FM transmitter of the first electrode device and the FM transmitter of the second electrode device transmit FM signals at different frequencies.

18. The biosignal measurement system of claim 16, wherein the FM transmitter comprises a voltage control oscillator and the FM receiver comprises a phase locked loop.

19. The biosignal measurement system of claim 18, wherein the adjustment circuit comprises an operational amplifier configured to adjust at least one of an amplification condition or an offset to match voltage-frequency characteristics between the voltage control oscillator and the phase locked loop.

20. The biosignal measurement system of claim 16, wherein at least one of: communication between the first and second electrode devices, or communication between the biosignal generation device and the right-leg drive device, uses the human body as a communication channel.
